(19) United States
(12) Patent Application Publication
(10) Pub. No.: US 2010/0184056 A1
(43) Pub. Date: Jul. 22, 2010

(54) Sample collection and measurement in a single container by back scattering interferometry

(75) Inventors: Scot Weinberger, Montara, CA (US); Darryl J. Bornhop, Nashville, TN (US); Stephen Dotson, Montara, CA (US); Bruce J. Richardson, Los Gatos, CA (US); Daniel Chang, Danville, CA (US)

Correspondence Address: Quine Intellectual Property Law Group, P.C., P.O. Box 458, Alameda, CA, 94501, US

(73) Assignees: Molecular Sensing, Inc., Montara, CA (US); Vanderbilt University, Nashville, TN (US)

(21) Appl. No.: 12/655,899

(22) Filed: Jan. 8, 2010

Related U.S. Application Data
(60) Provisional application No. 61/144,054, filed on Jan. 12, 2009.

(51) Int. Cl. C12Q 1/68; G01N 33/53; C12M 3/00

(52) U.S. Cl. 435/6; 435/7.1; 435/287.1; 435/287.2

(57) ABSTRACT

This invention provides a device and method for collection and analysis of heterogeneous samples in a single sample container by back scattering interferometry. The sample container is configured to allow collection of a heterogeneous sample, such as blood, from a subject, separation of insoluble materials, such as blood cells by, for example, centrifugation, and mounting on a back scattering interferometer for analysis. In certain embodiments the container is a capillary tube and the interferometer comprises a mounting to hold the capillary tube in position for analysis. The device and method allow point-of-care analysis of samples.

### CROSS-REFERENCE

This application claims the benefit of the priority date of U.S. provisional patent application 61/144,054, filed Jan. 12, 2009.

### STATEMENT AS TO FEDERALLY SPONSORED RESEARCH

This invention was made with Government support under Grant No. R01 EB003537-01A2 awarded by the National Institutes of Health. The Government may have certain rights in this invention.

### BACKGROUND OF THE INVENTION

Back-Scattering Interferometry (BSI) is a highly sensitive refractive index (RI) detection technology that utilizes an illumination source, a fluidic micro-channel, and a detector. A fringe pattern, a series of bright and dark spots, is created by positive and negative interference of the light on the fluidic channel. The shift in these fringes corresponds to a change in RI. When biomolecules, such as proteins, DNA, RNA, or some molecules, such as drugs, toxins, xenobiotics, allergens, and so on, interact with each other or with other targets, a BSI binding signal is created, resulting in a measured alteration in refractive index. BSI molecular interaction measurements can be performed in a homogeneous manner (free solution approach or untethered approach in which none of the interactors are physically bound to a solid support) or in a heterogeneous manner (tethered approach in which at least one of the interactors is bound to a solid support). Applications of BSI as well as its technical basis have been well described by Bornhop et al.

The elegant simplicity of BSI technology lends itself well as a platform for many applications, including in vitro diagnostics. Moreover, the platform can ideally be engineered to provide a near patient (NP) or point-of-care (POC) solution for testing of a variety of clinically relevant conditions including but not limited to: determination of serological titer (reactive titer), diagnosis of infectious disease, diagnosis of organic/metabolic disease, detection of cancer, detection of drugs of abuse, dose monitoring for a given pharmacologic regimen, rapid detection of sepsis, as well as rapid detection of cardiovascular distress. These assays can be performed using protein-protein (as with antibody-antigen tests), protein-DNA, protein-RNA or DNA-DNA interactions. Moreover, diagnostic assays which employ specific amplification of target nucleic acid sequence, such as polymerase chain reactions, may also be monitored by BSI, with or without the specific use of binding or hybridization.

A major challenge in near-patient or point-of-care applications is the means by which whole blood samples can be rapidly processed and then analyzed to provide a timely answer to enable an expedited diagnosis and rapid execution of the indicated therapeutic strategy. Additionally, clinical samples must be processed in a manner that does not cause artifactual alteration of the specimen, which in turn creates pre-analytical bias in the applied clinical test, resulting in compromised levels of performance (reduced sensitivity, reduced specificity, and concomitant reduction in positive/negative predictive value). Moreover, sample processing must be in line with current medical protocols and consistent with assay reimbursement demands of today's medical enterprise. Speed of analysis is also an important consideration in NP/POC applications. Often it is desirable to make a determination on-site, or as rapidly as possible as is the case when using makers of cardiac events or for determining contamination/infection by a biowarfare agent. Finally, in some cases it is necessary for the result to be quantitative, while in others just determining presence can guide the therapeutic intervention. The constraints encountered for performing assays on a low-resource or field setting are similar to those for a POC analysis. Sample processing must be limited, the assay must have an internal reference or calibration and the operational parameters of the assay should lead to a reliable and rapid result.

### SUMMARY OF THE INVENTION

In one aspect this invention provides a method comprising: a) introducing a sample into a container, wherein: i) the sample comprises a liquid and insoluble material; and ii) the container is adapted for analysis of the liquid therein by back scattering interferometry; b) separating the liquid from the insoluble material serum within the container; and c) analyzing the liquid while in the container by back-scattering interferometry. In one embodiment the sample is blood. In another embodiment the liquid is serum or plasma. In another embodiment the container comprises a solid substrate comprising a channel into which the sample is introduced. In another embodiment the container contains an anti-coagulant. In another embodiment the container contains a reagent for an assay for an analyte in the sample. In another embodiment the reagent is a binding partner for the analyte. In another embodiment the reagent is immobilized to an internal surface of a compartment of the container. In another embodiment the internal surface is patterned so that the reagent is attached only to a portion of the surface and that portion is positioned in a sensing area that is interrogated by a laser during back scattering interferometry. In another embodiment the method comprises detecting position of a fringe pattern generated at a location where the reagent is immobilized and at a location where the reagent is not immobilized and comparing the positions. In another embodiment the reagent is an enzyme. In another embodiment the assay is PCR and the container contains amplification primers, polymerase and nucleotides. In another embodiment the assay is a ligation assay and the container contains a ligase. In another embodiment a plurality of reagents are immobilized at different specific locations on an internal surface of the container. In another embodiment the reagent is free for entering solution upon introduction of a liquid sample into the container or a compartment thereof. In another embodiment the container is a chip. In another embodiment the container is a tube. In another embodiment the tube comprises borosilicate, fused silica or plastic. In another embodiment the tube has an internal diameter of 0.05 mm to 2.0 mm. In another embodiment the tube has a bore with at least one flat face. In another embodiment introducing comprises collecting blood from a subject into a capillary tube and sealing at least one end of the capillary tube. In another embodiment the separating comprises centrifuging the capillary tube. In another embodiment introducing comprises providing a capillary tube fitted on one end with a holder comprising a hole that securely fastens to the end of the capillary tube and a well for accepting liquid and passing it into the capillary tube. In another embodiment the method further comprises incubating the sample or agitating the sample after introduction. In another embodiment separating comprises centrifugation or sedimentation. In another embodiment analyzing comprises detecting the presence of an analyte in the liquid. In another embodiment analyzing comprises engaging the container with a holder of a back-scatter interferometry device in a position so that the container can be interrogated by a laser of the device. In another embodiment analyzing comprises comparing a signal received from the liquid with a signal received from a control liquid. In another embodiment analyzing comprises simultaneously illuminating the liquid and a control liquid with a single beam from the coherent light source and comparing the signals. In another embodiment back-scattering interferometry comprises directing a coherent light beam at the fluid in the container and detecting back scattered light. In another embodiment the coherent light is laser light.

In another aspect this invention provides a method comprising performing back-scattering interferometry analysis on a sample wherein the sample is stationary at the time of analysis.

In another aspect this invention provides a method comprising: a) collecting a liquid sample from a subject into a sample collection container containing a compartment adapted for analysis by back-scattering interferometry; and b) analyzing the liquid while in the sample collection container by back-scattering interferometry.

In another aspect this invention provides a device comprising: a) a coherent light source; b) a sample container for receiving a sample, wherein the container is configured for analysis of a sample therein by back-scatter interferometry when interrogated by coherent light from the coherent light source, and wherein the container is further configured to prevent flow of a sample in the container during analysis; and c) a detector to detect back-scattered light. In one embodiment the container is configured to centrifuge a sample contained in the container when the container is not engaged with the device. In another embodiment the container comprises at least one stop or valve that prevents fluid flow. In another embodiment the reagent is immobilized on an internal surface of the container. In another embodiment the container comprises a reagent for performing an assay on an analyte. In another embodiment the reagent is immobilized on an internal surface of the container. In another embodiment a plurality of different reagents are immobilized at different specified locations on an internal surface of the container. In another embodiment the container, e.g., an inside surface, is patterned with areas to which the reagents are immobilized and at least one area to which no reagent is immobilized. In another embodiment the reagent is immobilized by photolytically cleaving a protecting group attached to the surface to expose a reactive group and coupling the reagent to the reactive group. In another embodiment the reactive group is an isocyanate. In another embodiment the reagent is free for entering solution upon introduction of a liquid sample into the container. In another embodiment the coherent light source comprises a laser. In another embodiment the device further comprises a centrifuge adapted to engage the container, to centrifuge the container while engaged, and to position the container for analysis. In another embodiment the container contains a channel and is adapted to receive a sample into the channel through capillary action, wherein at least one end of the channel is closed when a sample is collected to allow centrifugation. In another embodiment the container comprises a hematocrit tube. In another embodiment the container comprises a chip. In another embodiment the device further comprises a second holder adapted to engage a second container in a position to be interrogated by an undivided beam of coherent light from the light source. In another embodiment the containers are positioned with respect to one another so that the detector detects the fringe pattern from each container distinctly.

In another aspect this invention provides a kit comprising: a) a device comprising: i) a coherent light source and ii) a holder adapted to engage a container in position so that a liquid in the container can be interrogated by coherent light from the coherent light source to produce a back scattered light; and iii) a detector configured to detect the back scattered light; and b) a container comprising a reagent for assaying for an analyte in the container, wherein the container is adapted to engage the holder. In another embodiment the container is a hematocrit tube. In another embodiment the container is a capillary tube of fixed length. In another embodiment the reagent comprises a binding partner for the analyte. In another embodiment the binding partner comprises a polynucleotide, a polypeptide or a small organic molecule. In another embodiment the reagent is immobilized on an internal surface of the container or a compartment thereof. In another embodiment the reagent is locally immobilized on the internal surface of the photonicly probed region of the compartment or container. In another embodiment the reagent is free for entering solution upon introduction of a liquid sample into the container. In another embodiment the kit further comprises c) a sealant for sealing or a cap for closing an opening in the container.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: The schematic of the capillary tube 101 shows a typical capillary. The three lines show the volume of the bulk/volume of surface ratios for a 5 nm layer thickness 102, a 10 nm layer thickness 103, and a 25 nm layer thickness 104.

FIG. 2 depicts a flow diagram of a BSI system. A laser 201 produces a beam that passes through a beam splitter 202 to create two beams. A beam splitter is optional but useful for comparing first and second samples. These two beams impinge onto a chip 203. The two channel chip allows for the injection of samples and controls 204. The liquid that is injected passes through the chip 203 and then is collected as waste 205. The interaction of the beams and the channels creates fringe patterns 206. These two fringe patterns 206 are directed onto a camera 207. The data acquired from the camera 207 is converted into a digital image 208. Initially, the program is started in setup mode 209, which allows the user to select the fringes to be analyzed and define the parameters of the analysis 210. Once setup mode 209 is turned off, the digital image 208 is passed to an algorithm 211 that calculates shifts in the fringe pattern 206. This output is split 212 to a real time output display 213 and is also written to a temporary file 214. At any time the user can save the data 215, which then writes the data to a permanent file 216.

FIG. 3 depicts an example of an algorithm. The digital image 301 and user input for fringes and parameters 302 are used to select the regions from the digital image 301. The camera allows for two regions to be selected (sample and control for most applications). This data is then split 304 into different algorithms, a new algorithm 305 and an example algorithm 306 (that will be expanded on to demonstrate how calculations are performed), as the program allows for multiple analyses to be performed simultaneously. The data from these algorithms are written to a temporary file 307 and are displayed 308 in real time. The Fast Fourier transform (PEI) is an excellent example as FFT is used in many techniques, such as FTIR. The output from 303 is passed to the algorithm as an array of numbers that describe the fringe pattern 309. A FFT is performed 310 and then the algorithm locks in on the spatial frequency 311 as defined by the user in 302. The output of this algorithm is the phase of the spatial frequency 312. This algorithm is applied to both the sample and reference data simultaneously.

FIG. 4 depicts the Gaussian Fit Algorithm. The array of numbers describing the fringe pattern 401 and an array of numbers describing a reference fringe pattern 402 are used in a cross correlation 403. A Gaussian fit 404 of the highest peak of the cross correlation is calculated. The center of the Gaussian fit is calculated and then output 405.

FIG. 5 depicts the Gaussian fit with a Hamming window. Both the array of numbers describing the fringe pattern 501 and an array of numbers describing a reference fringe pattern 502 have a hamming window applied to the fringe pattern 503, then a cross correlation is performed 504. The Gaussian fit of the highest peak is calculated 505 and the center of the Gaussian fit is calculated and output 506.

FIG. 6 depicts the Sinusoidal Data Correction. After the algorithm 601 the data is split 602 to an output 603 and written to file 604. Once all the data is collected, the maximum and minimum values are found 605. Each value 606 is divided by (max−min)/2 then the new maximum is located 607. The (1−max) is added to each array value 608 (center around 0). The arcsine of each array value is taken 609. Using user selected regions 610, switch between Point B−Point A+previously calculated value and Point A−Point B+previously calculated value 611. Multiply each array value 612 by the same value that was used to divide in 606. Then write the data to file 613.

FIG. 7 depicts a design for a large tube device. The top piece 701 is a holder/well that is used to hold the tube and allow for injection of liquids. The tube 702 can be inserted into a droplet of sample 703. Sample is then drawn into tube 702 via capillary action. Sample 703 can be of various origin, including a droplet of blood created via a finger stick.

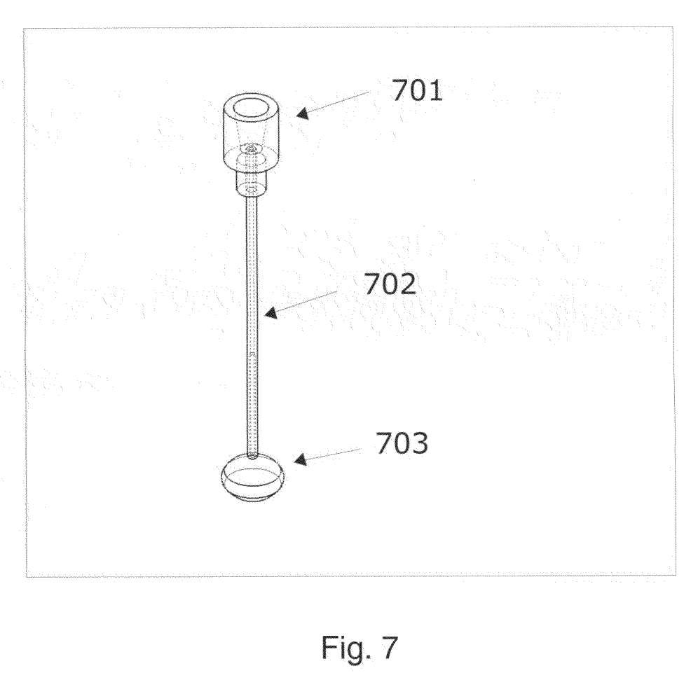

FIG. 8 shows a modified design for a large tube device. The top piece 801 is a holder/well that is used to hold the tube and allow for injection of liquids. The tube 802 is a capillary and can be sealed at the end by a seal cap 803.

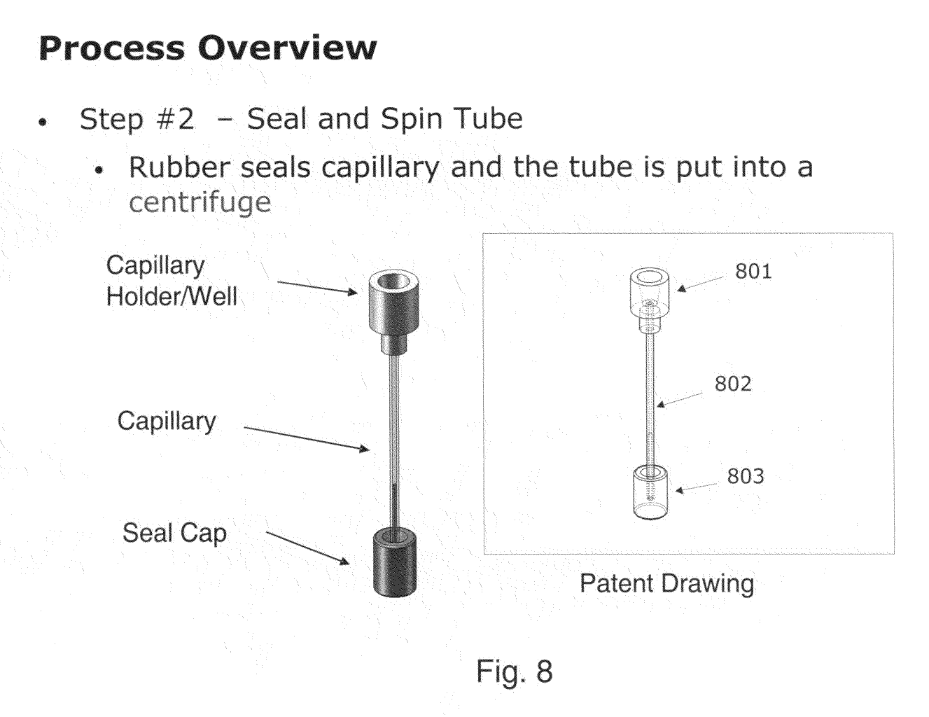

FIG. 9 shows a modified design for a large tube device. The piece 901 is a holder/well that is used to hold the tube and allow for injection of liquids. The tube 902 is a capillary and can be sealed at the end by a seal cap 903. A new cap 904 allows for the sealing of the top capillary holder/well 901.

FIG. 10 depicts a holder/instrumentation for the tube design of this invention. A heat sink 1001 is attached to a thermal electric cooler (Peltier) 1003 which is up against a holding block 1005. This allows for the holding of the tube 1002. A sliding clamp 1004 is used to hold the tube 1002 in place. An incoming beam 1006 is directed onto the tube 1002 and a fringe pattern is created 1007.

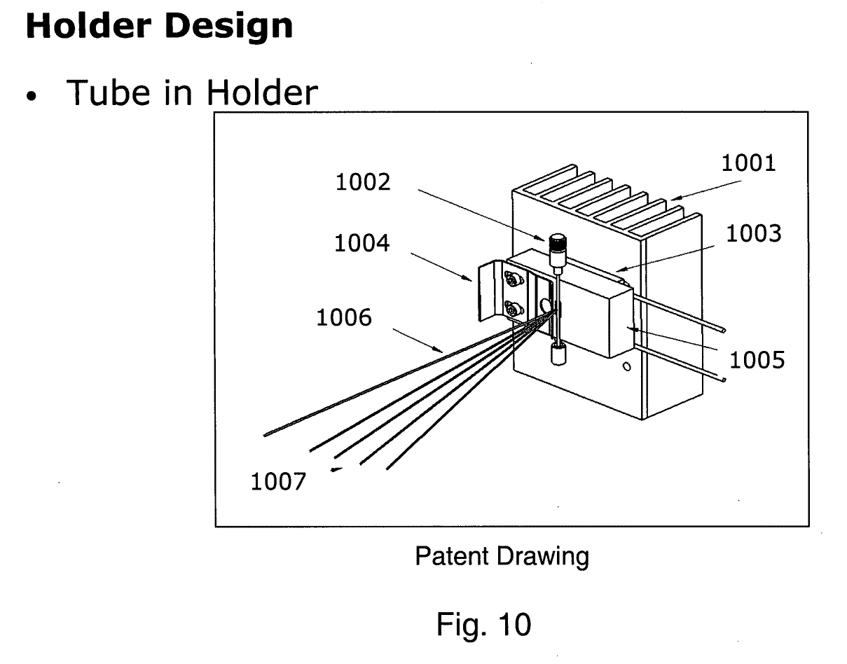

FIG. 11 depicts a holder/instrumentation for containers of this invention, e.g., the designs in FIGS. 7-9, before the capillary is installed. A heat sink 1101 is attached to a thermal electric cooler (Peltier) 1103 which is up against a holding block 1105. This allows for the holding of the tube 1102. A sliding clamp 1104 is used to hold the tube 1102 in place. An incoming beam 1102 is directed onto the block 1105.

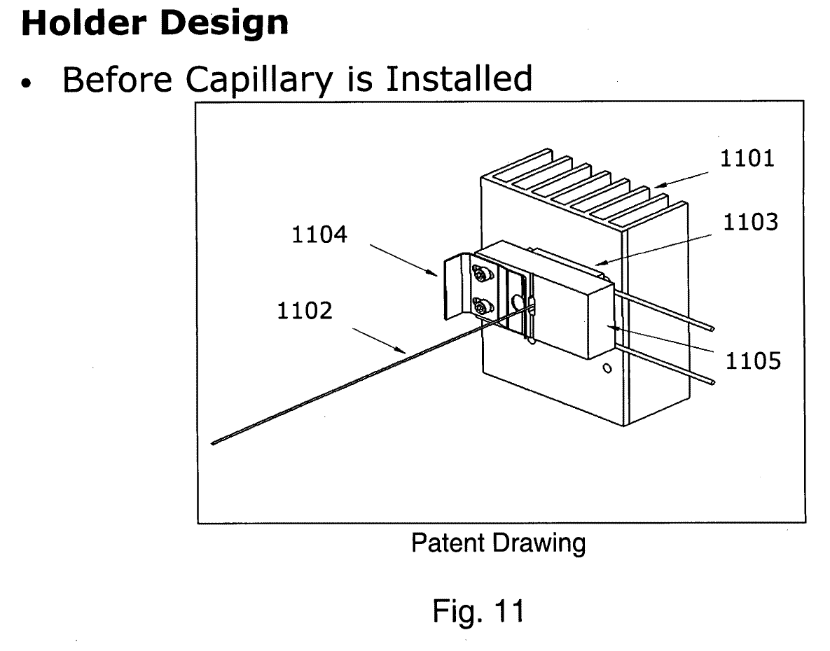

FIG. 12 depicts a holder/instrumentation for a capillary design of this invention. A heat sink 1201 is attached to a thermal electric cooler (Peltier) 1203 which is up against a holding block 1205. This allows for the holding of the capillary 1202. A sliding clamp 1204 is used to hold the capillary 1202 in place. An incoming beam 1206 is directed onto the capillary 1202 and a fringe pattern is created 1207.

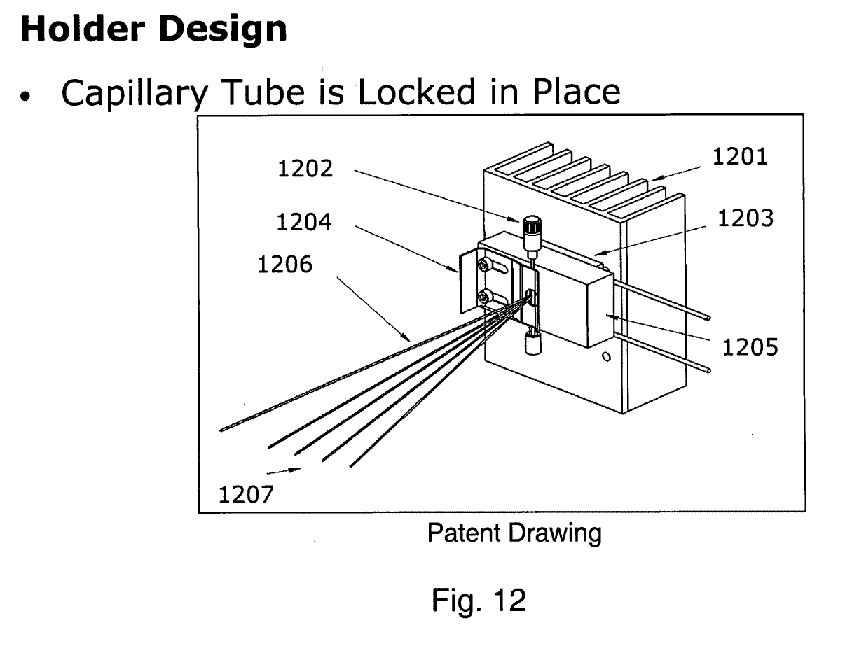

FIG. 13 depicts an exemplary full BSI device configured for analyzing a blood sample. The clamp 1301 holds the laser 1302 in place. The translation 1303 moves the laser 1302 to the left and right to allow alignment. The beam 1306 hits the tube 1308 and creates a fringe pattern 1305. A mirror 1307 is used to direct the fringe pattern 1305 onto camera 1304. The translation 1309 allows for the alignment of the camera 1304. The tube 1308 sits in a holder 1311 that is temperature controlled by a thermal electric cooler 1312 and a heat sink 1313 is used to dissipate the temperature difference. The angle adjustment 1310 is used to align the tube 1308.

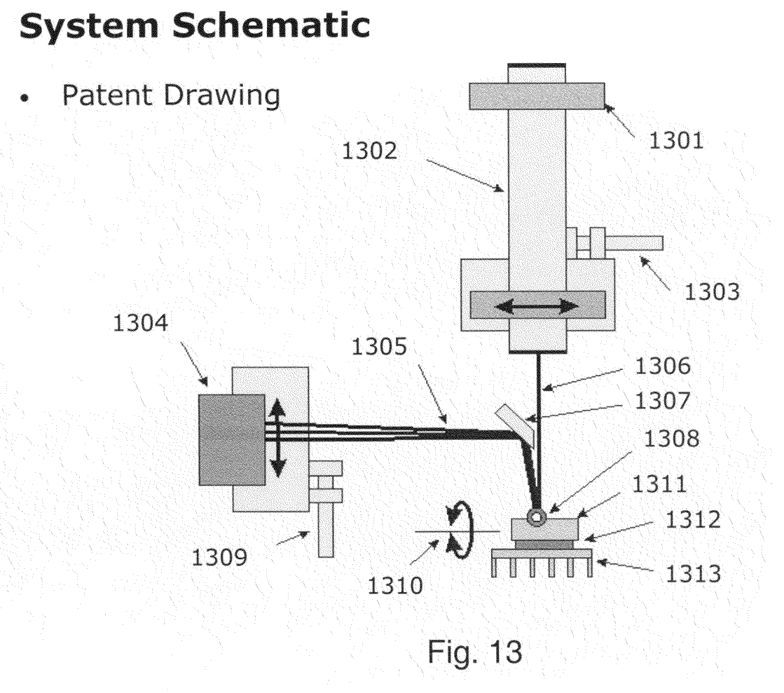

FIG. 14 shows a system for sample analysis in flowing streams.

### DETAILED DESCRIPTION OF THE INVENTION

This invention provides a method in which a heterogeneous sample, such as a blood sample, can be collected and analyzed by back-scattering interferometry in the same container in which it was collected and without the need to transfer the sample from a collection container to an analysis container. The invention involves using, as the collection container, a container that also is adapted for analysis by back scattering interferometry (“BSI”). In order to allow the collection of a heterogenoeus sample, such as blood, the container typically will include a bore, such as a channel or compartment, that opens on two sides of the container to allow flow of the sample into the container, for example by capillary action. The openings can be stopped after collection of the sample to maintain the liquid in place. In one embodiment, the container is a capillary tube, such as a hematocrit tube.

After collection, insoluble materials can be separated from the aqueous material by, e.g., centrifugation or settling. The container is loaded onto the interferometer so that the coherent light can pass through the container into the liquid and reflect back an interference pattern that is detected by a detector and analyzed, typically, by computer.

### I. Sample

The samples used in this invention can be any liquid sample. Typically the sample will be a heterogeneous sample that includes a solvent, soluble or suspended materials, and insoluble materials. In particular, the fluid can be a biological sample, for example, saliva, blood, urine, lymphatic fluid, prostatic or seminal fluid, milk, lymph, cerebrospinal fluid, synovial fluid, vitreous humor, aqueous humor, mucus, vaginal fluid or semen. The liquid also can be derived from biological materials, such as cell extracts, cell culture media, fractionated samples, or the like. In one embodiment, the sample is blood or a blood fraction, such as serum or plasma. Blood is an aqueous solution. It contains soluble or suspended materials including electrolytes and biomolecules such as polypeptides, polynucleotides, polysaccharides, lipids, proteins, glucose, clotting factors, mineral ions, hormones, steroidal compounds, etc. It also includes insoluble materials such as blood cells, cellular debris, and clots. Plasma is blood from which the cells have been removed. Serum is blood plasma without fibrinogen or the other clotting factors. As shall be discussed, the sample can be collected in the same container to be used in the back scattering interferometry analysis, and the insoluble materials can be separated therein.

### II. The Container

The container used in this invention is adapted for use in back scattering interferometry. It also is adapted to collect a fluid sample and to allow separation of insoluble material from the fluid while in the container.

The container is adapted to generate a backscatter fringe pattern when filled with liquid and interrogated with an unfocused coherent light source, such as a laser beam. Factors that influence the ability to create such a pattern include the relative refractive indices of the substrate that forms the container and the liquid within, as well as the shape of compartment in which the liquid is contained and the light source strikes.

A. Container Material

The container should be made of a material that has a different (e.g., higher) refractive index than the sample inside. The container can be formed of any suitable optically transmissive material, such as glass, quartz, borosilicate, silica (e.g., fused silica) or a polymeric material, e.g., a plastic such as polyacrylate, cyclic olefin copolymer, polydimethyl siloxane, polycarbonate, and polymethyl methacrylate. The container can be mounted on or brought into thermal contact with the thermal subsystem of the temperature control unit. The thermal subsystem can be, for example, a peltier device.

B. Compartment Shape

The container will have an internal compartment that can hold the sample. Typically, the compartment will take the shape of a bore. The bore may be have a curved cross section that is, for example, circular, hemicircular, elliptical or substantially these shapes. Backscatter fringe patterns are easily produced when the substrate includes a compartment having curved or angular walls through which the light passes to reach the sample. However, useful backscatter patterns also have been produced with rectangular shaped compartments.

In certain embodiments, the compartment takes a long, thin shape, such as a channel, column, cylinder or tube, e.g., a capillary tube.

The container also is adapted to receive a liquid sample. In certain embodiments, the container is adapted to function as the collection unit of the sample from its primary source, e.g., a subject organism. For example, the container can be adapted to receive blood. One method of collecting liquid into a container is by capillary action. For example, the compartment could be in the shape of a long, thin channel. It also can take the shape of a chip having a capillary channel in it. A preferred arrangement for collection of a fluid sample from a subject is a container having a compartment that opens in two different places on the container. For example, the container can comprise a channel or tube that opens at two ends of the container. For example, the container can be a capillary tube or a hematocrit tube, or a chip comprising a channel that opens at different sides of the chip. The chip also could comprise tube-shaped extensions at a collection end of the tube.

The container can take the shape of a capillary tube or micro-hematotcrit tube. The tube can be, for example, approximately 75 mm long, with fire-polished ends that can easily be sealed if desired. Tube can be coded with a red band to designate heparin coating. It can contain at least 2 U.S.P. units of cation-free ammonium heparin. It can have an I.D. is 1.1 mm to 1.2 mm with a wall of 0.2 mm±0.02. The volume of the compartment can be between 100 nanoliters and 1000 microliters (10 milliliters), between 1 microliter and 1 milliliter, between 10 microliters and 1 milliliter or between 50 microliters and 250 microliters. Furthermore the tube can have dimensions as follows: Outside diameter 0.75 to 2.0 mm, inside diameter from 0.05 to 1.5 mm.

The average internal diameter of the compartment can range, for example, from about 0.05 mm or about 0.065 mm to any of about 0.25 mm, about 1.5 mm or about 2 mm, e.g., about 0.065 mm to about 0.25 mm. The cross sectional area of the compartment can range, for example, from about 1.9×10⁻³ mm² or about 3×10⁻³ mm² to any of about 0.05 mm², about 1 mm² or about 3.2 mm², e.g., about 3×10³ mm² to about 0.05 mm².

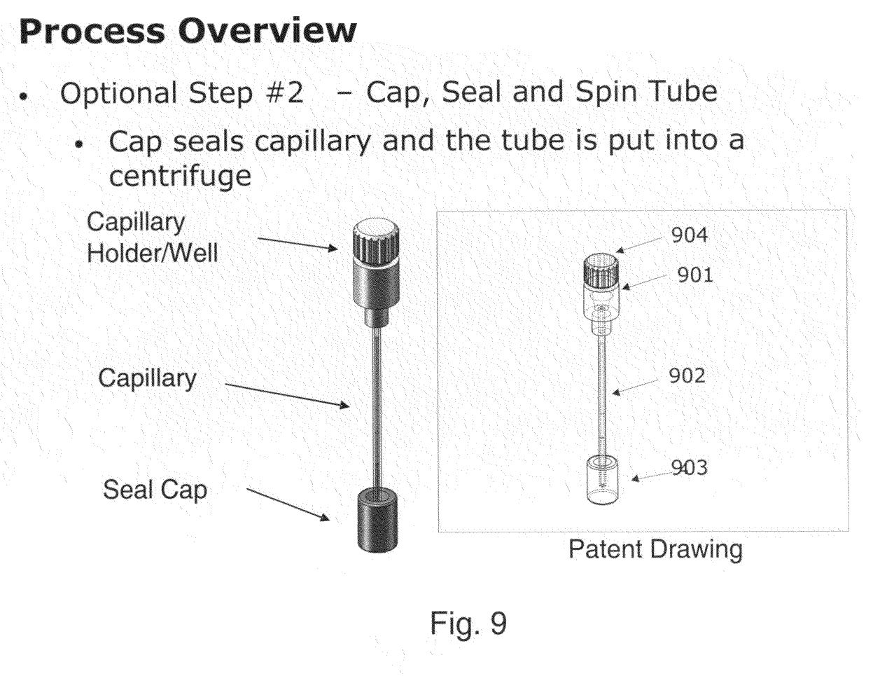

When adapted for the collection of a heterogeneous sample, such as blood, the container also should be sealable or closable on at least one open end of the compartment or channel. One way of closing a channel is by providing a cap that fits either end and prevents sample from leaving the container. (See, e.g., FIG. 9, items 904, 901 and 903.) Another method involves inserting a plug into the opening. For example, one can introduce a liquid sample, such as blood, into a container, such as a capillary tube, by capillary action and then push one end of the tube into a malleable material such as clay to close off the end. Other methods include compressing the end of a tube made of a compressible material, placing a solid material over the end of the opening (e.g., pressing the open end against the material), closing a lid on the opening or opening or closing valves.

In certain methods, BSI provides data in real time by flowing a liquid through a chip and detecting changes in RI over time. In embodiments of the present invention, the container is configured to prevent flow of the sample. This can be accomplished by, for example, stopping one or both ends of the container while it is in the interferometry device. The container also may contain a valve that allows flow in one direction only. In this case, the valve is configured to remain closed during analysis, thereby preventing liquid flow.

The containers can include within them materials, such as lyophilized powders, that contain reagents with which the fluid sample reacts. These materials can be frosted on the inside walls of the container, or free to move within the tube.

In one embodiment, the reagents can be an anticoagulant, to prevent clotting of blood. Anticoagulants include, for example, heparin, hirudin, EDTA, citrate and oxalate. In another embodiment, the reagent can be a material that accelerates clotting of blood. Such reagents include, for example, thrombin. In other embodiments, the reagent can comprise one or more protease inhibitors.

In certain embodiments the analyte is detected as a result of its binding to a binding agent. In this case, the binding agent for an analyte in a sample that one is testing for can be immobilized on the wall of the compartment (heterogeneous assay) or allowed to remain free in solution after the sample is added (homogenous assay). Binding partners include, for example, antibodies and antibody-like molecules, receptors, nucleic acids (e.g., oligonucleotides). In another embodiment, the reagent can be an enzyme or enzyme complex (mixture) which catalyzes a enzymatic reaction which can degrade sample components such as cells, cell fragments, and/or biomolecules. In another embodiment the reagent could be an enzyme or enzyme complex (mixture) which catalyzes the creation of new biomolecules arising from the fusion of biomolecular species (such as a ligase) or replication-amplification of biomolecular species, as is the case in polymerase chain reactions. Moreover, the surfaces of the sample container could be coated with a material to minimize unwanted interactions with the walls of the container. Such surfaces would include polymeric coatings, such as dextran, Teflon, polyethylene glycol, etc. Furthermore, the surfaces of the container could be coated with biospecific reagents for selective capture of target analytes or selective enzymatic modification of target analytes as described above.

1. Patterned Surface

In certain embodiments, the container is provided with patterned internal surface. Such a surface allows a binding reagent to be bound only in the sensing area to be probed by the light source. In this way reagent is not wasted and does not deplete the sample of analyte. Methods of making a pattern on the inside wall of a tube are well known in the art. See, for example, Dendane et al., Lab Chip 2008, 8:2161. For example, the binding agent can be patterned in the form of a ring around the inside of the compartment. Different binding agents can be bound to different specific areas of the compartment surface so that more than one assay can be performed in a single compartment. Thus, the compartment can have a series of rings, each ring having a different reagent (e.g., binding agent) bound to the wall. The rings can be separated with rings having no bound material and that can act as control regions. Other areas of the surface of the compartment will have no binding agent attached and can serve as internal control regions.

In general, the internal surface is provided with photoreactive groups, such as a benzophenone. The area where the binding or linking molecule is to be attached is photoirradiated to expose the functional groups. In one embodiment, reactive aminooxyl groups are masked with a photocleavable protective group, e.g., 2-(2-nitrophenyl)propyloxycarbonyl. After irradiation, the aminooxy groups are coupled to molecules having available aldehydes. In another embodiment, the inner surface is coated with a compound having an isocyanate group, such as toluene diisocyanate or 1,4-cyclohexane-diisocyante. One of the isocyanate groups is protected by, for example, o-nitrobenzyl alcohol. Upon irradiation by UV light, the protective group is released, revealing an amino group. The amino group can then react with aldehyde-functionalized compounds or biomolecules to facilitate covalent surface immobilziation. Alternatively, the molecule attached can have another functional group, such as N-hydroxy-succinate, N-hydroxy-maleate aldehyde, expoxy or carbodiimide. See, for example, U.S. application Ser. No. 12/587,112 (filed Sep. 30, 2009, Chang). This embodiment results in improved quantitative and qualitative detection limits, linear dynamic range, and overall dynamic range.

C. Container Diameter

It has been found that increasing the volume-to-compartment surface area increases the sensitivity of measurements for detection of molecular interactions in free solution as well as the fidelity of the measurement in free solution, by minimizing unwanted signal that may arise due to anecdotal binding to the internal surface of the compartment. Accordingly, for free solution analyses, large bore tubes, such as hematocrit tubes, are expected to provide greater detection sensitivity and superior analytical performance when compared to smaller bore diameters.

For a cylinder of radius r and length l, the circumference is c, the area of the circle cross-section is a, the surface area of the cylinder is S and the volume of the cylinder is V:

c=2π·r

a=π·r²

S=c·l=2π·r·l

V=a·l=π·r²·l (1)

The ratio of volume to capillary surface area is r/2,

$$\frac{V}{S} = \frac{π \cdot {r}^{2} \cdot l}{2 π \cdot r \cdot l} = \frac{r}{2} \qquad ( 2 )$$

The ratio of bulk volume to surface layer volume is:

$$\frac{{V}_{bulk}}{{V}_{layer}} = \frac{π \cdot {( r - t )}^{2} \cdot l}{( π \cdot {r}^{2} - π \cdot {( r - t )}^{2} ) \cdot l} = \frac{{( r - t )}^{2}}{t \cdot ( 2 r - t )} \qquad ( 3 )$$

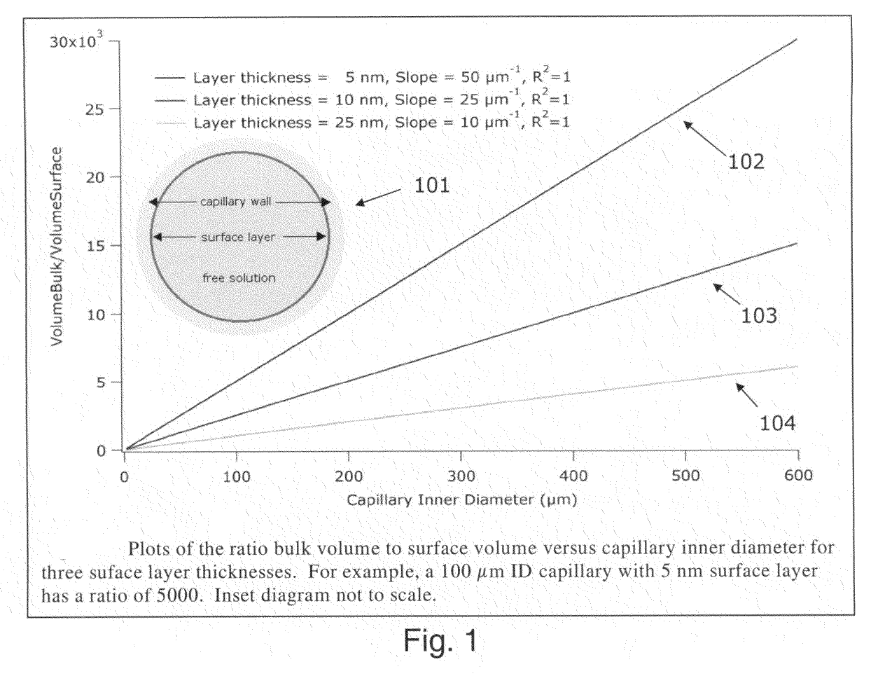

FIG. 1 plots this exact ratio versus capillary inner diameter (=2r), for three layer thicknesses. Note the vertical axis maximum is 30,000 for a 600 μm ID capillary with 5 nm surface layer. Surface layer thickness of 5 nm is the thickest observed. The 10 nm and 25 nm layer thicknesses are unrealistic, but are included to show the trend.

To simplify Equation 3, we use the fact that r>>t (50 μm>>5 nm, for example),

$$\frac{{V}_{bulk}}{{V}_{layer}} \approx \frac{( r - 0 )}{t \cdot ( 2 r - 0 )} = \frac{{r}^{2}}{t \cdot 2 r} = \frac{ID}{4 t} \qquad ( 4 )$$

The validity of this approximation is shown in FIG. 1, where a linear least-squares fit of the exact ratio (Eq 3) is proportional to capillary ID, and inversely proportional to 4t. Equation 4 can be used to calculate the volume ratio for any capillary ID and surface layer thickness, as long as r>>t is true, which is the case in real experiments.

The trend, and conclusion, of this graph is that the vastly predominant fraction of signal from capillary BSI measures RI of the free (bulk) solution, not the surface layer. For a common 100 μm ID capillary with a surface layer of 5 nm thickness, Vbulk/Vlayer=5000. This of course assumes the density of the solute species is the same for the bulk and any wall adhered species.

This result indicates that for each surface-bound entity there are five thousand entities in the free solution for every species attached to the surface. Since the RI signal should be proportional to the number of entities in the laser-capillary interaction volume, the signal from entities in free solution would be 5000 times the signal from surface-bound entities. It should be noted that this observation assumes that there is no unique surface enhancement factor as a consequence of the laser wall interaction. Note that it also assumes an equal distribution of rays (beam solution interaction) for solutes near the surface and for those far from the surface. It could be possible that the whispering gallery mode effect leads to a disproportionate sampling of the surface solutes compared to the bulk. In any event clearly for larger diameter tubes the bulk signal dominates.

III. Separation of Insoluble Material from Soluble Material

Once collected, heterogeneous liquids, such as blood or urine, can be separated before analysis. Several methods of separating soluble from insoluble materials are contemplated. In one method, the tube can be stood on one end, and the insoluble material can be allowed to precipitate out of solution. In another method, the container can be centrifuged to precipitate the insoluble materials. Centrifuging blood will cause the erythrocytes to settle to the bottom of the tube, the white cells (also called the “buffy coat”) to settle on top of the red cells and the plasma to lay on top. Any centrifuge adapted to spin the container in which the sample has been collected can be used. For example, horizontal centrifuges for hematocrit determination are well known in the art.

Once the insoluble materials are separated, the remaining solution is analyzed in the container. For example, the portion of the compartment, e.g., the tube that contains the solution functions as the sensing area that is interrogated by the coherent light source.

### IV. Back Scattering Interferometry

Once prepared, the sample is ready for analysis by back-scattering interferometry.

A back-scattering interferometer typically comprises an optical assembly and electronics to analyze an optical signal. The optical assembly can be mounted on an optical bench. Back-scattering interferometers are well known in the art. They are described, for example, in U.S. Pat. Nos. 5,325,170, 6,381,025; 6,809,828 and 7,130,060; International applications WO 2004/023115, WO 2006/047408 and WO 2009/039466; and U.S. patent publications U.S. 2006-0012800 and 2009-0185190.

The optical assembly comprises the following elements: First, a fluidic container having a compartment for holding a sample. A portion of the container in which the sample is contained functions as a sensing area or detection zone. Second, the optical assembly comprises a coherent light source positioned to direct a beam toward the sensing area, wherein the path of the beam defines an optical train and generates a back-scattering light pattern, also called an interference fringe pattern. Third, the optical assembly comprises a photodetector configured to detect the back-scattering light pattern. Typically, the instrument also will comprise a computer that converts the fringe pattern into a measure or indicator of refractive index. Optionally, the instrument comprises a temperature regulator that can maintain a stable temperature at least within the fluid during periods of measurement.

Several factors influence the generation of an interference pattern: Reflection, refraction and retardation (of the light beam). The coherent light beam should be large enough so that it passes across a non-flat surface from the container into the liquid. Accordingly, the compartment should comprise a curve or an edge (e.g., a corner) through which the light passes in order to generate a useful interference pattern.

1. Coherent Light Source

Examples of coherent light sources for use with the invention include, but are not limited to, a laser, for example a He/Ne laser, a vertical cavity surface emitting laser (VCSEL) laser, and a diode laser. The coherent light may be coupled to the site of measurement by known wave-guiding or diffractive optical techniques or may be conventionally directed to the measurement site by free space transmission. The coherent light is preferably a low power (for example, 3-15 mW) laser (for example, a He/Ne laser). As with any interferometric technique for chemical analysis, the devices and methods of the invention benefit from many of advantages lasers provide, including high spatial coherence, monochromaticity, and high photon flux. The beam can be directed directly to a sensing area on the fluidic chamber or to a minor that is angled with respect to the plane of propagation of the laser beam, wherein the mirror can redirect the light onto the sensing area. In another embodiment, the coherent light is preferably generated by a solid state laser source such as a light emitting diode or vertical cavity surface emitting laser (VCSEL), for which requisite beam characteristics of monochromaticity and beam coherence is achieved. In an embodiment, the coherent light source generates an easy to align collimated laser beam that is incident on a sensing area of the container for generating the backscattered light.

A coherent light source can be directed onto a sensing area of the container chip such that the light beam is incident on the compartment to generate backscattered light through reflective and refractive interaction of the light beam, as well as retardation of the light beam, with the sensing area interface and the sample. The backscattered light comprises interference fringe patterns including a plurality of spaced light regions, e.g., bands or spots, whose positions shift in response to the refractive index of the sample. These spatial shifts represent phase shifts in the interference pattern. Positional shifts in the interference pattern can then be detected by a photodetector and computed using a processor, such as a PC. For example, one can examine shifts in the light regions, e.g., bands, relative to a baseline or a reference value. The device can provide a signal (for example, positional shifts in the light bands) that is proportional to abundance of the analyte.

In an embodiment, the coherent light source generates an easy to align collimated laser beam that is incident on a sensing area of the container for generating the backscattered light. The backscattered light comprises interference fringe patterns that result from the reflective and refractive interaction, as well as retardation of the incident laser beam with the sensing area walls and the sample in the sensing area. These fringe patterns include a plurality of light bands whose positions shift according to the refractive index of the sample, for example, due to the composition of the sample. The photodetector can detect the backscattered light fringe pattern and, in combination with algorithms and methods and systems described herein, convert it into signals that can be used to determine the refractive index (RI), or an RI related characteristic property, of the sample. For example, the RI of a sample with a certain concentration of analyte in the sample can be slightly different than the RI of a sample where the analyte is present in the sample in a different concentration. A signal analyzer, such as a computer or an electrical circuit, can be employed to analyze the photodetector signals and determine the characteristic property of the sample.

B. Detector

A photodetector can be configured and incorporated into a device of the invention to detect a back-scattering light pattern from a sensing area on a container. The photodetector can detect a back-scattering light pattern generated from a sample in the sensing area of the chip, wherein the pattern is based on the contents and/or composition of the sample. In an embodiment, qualitative and quantitative measurements are performed by forming molecular complexes, such as antibody-antigen. Detection can be performed in a similar manner to an ELISA measurement, only a label on the antibody (in the case of an antigen based assay) is not used. In an embodiment, the photodetector detects a qualitative or quantitative value of an analyte in a liquid sample, for example, the amount of a specific antigen in a blood sample or host antibody titer towards a given antigen.

The photodetector can be one of any number of image sensing devices. It can capture an image, either linear or two-dimensional, of the fringe pattern. The photodetector can include a bi-cell position sensor, a linear or two-dimensional array CCD or CMOS camera and laser beam analyzer assembly, a slit-photodetector assembly, an avalanche photodiode, or any other suitable photodetection device. The backscattered light comprises interference fringe patterns that result from the reflective, refractive, and retardation interaction of the incident laser beam with the walls of the sensing area and the sample. These fringe patterns include a plurality of light bands whose positions shift as the refractive index of the sample is varied, for example, through compositional changes. For example, a sample in which two components bind to each other can have a different refractive index than a sample in which the two components do not bind. In an embodiment, the photodetector detects the backscattered light and converts it into one or more intensity signals that vary as the positions of the light bands in the fringe patterns shift. For fringe profiling, the photodetector can be mounted above the chip at an approximately 45° angle thereto. Fringe profiling can also be accomplished by detecting the direct backscatter. In an embodiment, the fringes can be profiled in direct backscatter configuration and direct them onto the camera which is at 90° from the beam, in this way, the packaged device can remain small while maximizing the resolution for measuring a positional shift, for example, the effect of angular displacement.

The photodetector can be a camera, such as a CCD camera. The camera captures the image of the fringe pattern. A CCD camera can typically collect from one to sixty images per second. The image can be projected on a monitor for visual analysis. For example, the monitor can be calibrated and/or the operator can visually detect changes in the fringe pattern over time. Alternatively, the image can be subjected to a variety of mathematical algorithms to analyze the fringe pattern. Examples of algorithms used to analyze fringe pattern are Fourier transforms, Gaussian fit with or without hamming window and sinusoidal correction.

In certain embodiments, the device comprises a back scattering interferometer and a centrifuge for centrifuging the sample container. For example, the centrifuge could be adapted to spin a tube that contains the sample, and position it so that the sensing area is in line with the optical train.

In another embodiment, the centrifuge and back scattering interferometer are separate devices, and the user or a robot transfers the sample tube from the centrifuge to the interferometer.

The intensity signals from the photodetector can be fed through an instrument control unit into a signal analyzer for fringe pattern analysis for determination of the refractive index or an RI related characteristic property of a sample in the sensing area of the microfluidic chip. The signal analyzer can be a computer (for example, a PC) or a dedicated electrical circuit. Preferably, the signal analyzer includes the programming or circuitry necessary to determine from the positional shift of the formed fringes, the RI or other characteristic properties of the sample to be determined, such as temperature or flow rate, for example.

C. Display and Analysis

The light collected by the photodetector, e.g., an image of a fringe pattern, can be displayed directly for visual analysis, for example by a monitor that displays a signal provided by the detector. Alternatively, the system can comprise a signal analyzer that converts data received from the photodetector into a value or values that are useful for further analysis.

The photodetector can detect the backscattered light fringe pattern and, in combination with computer algorithms, convert it into signals that can be used to determine a parameter of refractive index (RI), or an RI related characteristic property, of the sample. For example, the RI of a sample with a certain concentration of analyte in the sample can be slightly different than the RI of a sample where the analyte is present in the sample in a different concentration. A signal analyzer, such as a computer or an electrical circuit, can be employed to analyze the photodetector signals and determine the characteristic property of the sample. Positional shifts in the light bands relative to a baseline or a reference value can then be detected by a photodetector and computed using a processor, such as a PC. The device can provide a signal (for example, positional shifts in the light bands) that is proportional to abundance of the analyte. Preferably, the signal analyzer includes the programming or circuitry necessary to determine from the positional shift of the formed fringes, the RI or other characteristic properties of the sample to be determined, such as temperature or flow rate, for example. The parameter of refractive index can be, for example, the position of the bands on some scale of location. This position can be displayed as a number or as coordinate on a graph. For example, the coordinate on the Y axis can change over time on the X axis. The parameter can be quantitatively related to sample refractive index.

The signal analyzer can comprise a computer which, optionally, controls various aspects of the system. The computer functions to perform the calculations necessary to detect the fringe movement and output the data on the user interface. Moreover, the computer can function to store and retrieve method files that automate the performance of an assay or analysis, provide data analysis tools to determine binding profiles, qualitative measurements, and quantitative measurements, or provide a means to calibrate the system for total gain and output based upon a reference sample.

The computer can comprise memory configured to receive data about the back scattered light, such as images of the fringe pattern, captured from the photodetector. The computer also can comprise computer executable instructions in memory to manipulate the data, for example, methods according to this invention. The computer typically will comprise a processor for retrieving data and instructions from memory and for executing the instructions. The computer also can comprise input/output to receive data from the photodetector and to transmit the product of computer processing to peripherals such as display monitors.

The output of the computer can be displayed on a monitor in a form useful to the user. For example, the output can be displayed as a line on a graph, wherein the position of the line indicates the relative position of the fringe pattern. Alternatively, the output could be a binary indicator that indicates whether the position of the fringe pattern has shifted over some given period of time, or before and after an event (e.g., introduction of an analyte).

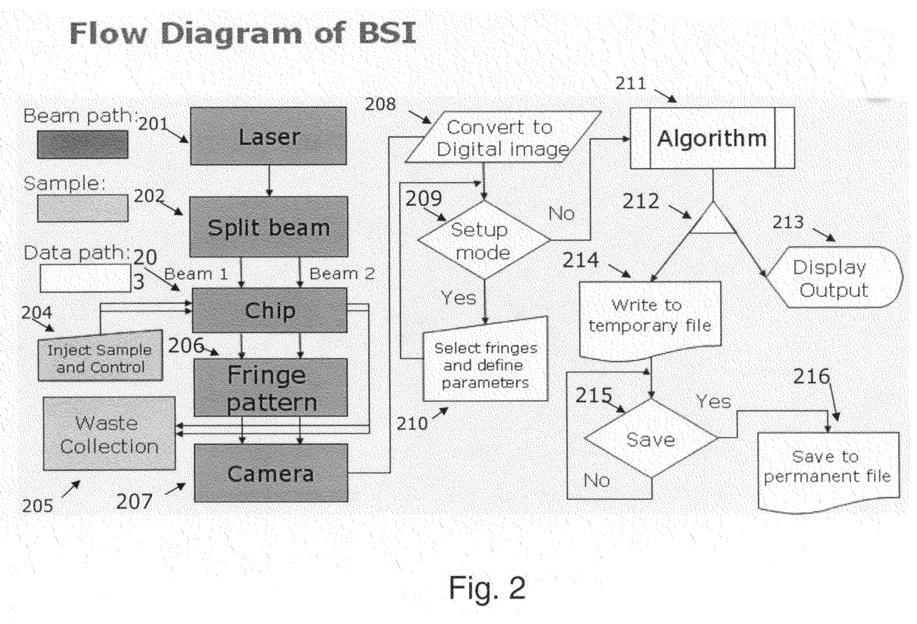

FIG. 2 depicts a flow diagram of a BSI system. A laser 201 produces a beam that passes through a beam splitter 202 to create two beams. A beam splitter is optional but useful for comparing first and second samples. These two beams impinge onto a chip 203. The two-channel chip allows for the injection of samples and controls 204. The liquid that is injected passes through the chip 203 and then is collected as waste 205. In an exemplary embodiment, the chip has two channels for the injection of samples and controls 204. The interaction of the beams and the channels creates fringe patterns 206. These two fringe patterns 206 are directed onto a camera 207. The data acquired from the camera 207 is converted into a digital image 208. Initially, the program is started in setup mode 209, which allows the user to select the fringes to be analyzed and define the parameters of the analysis 210. Once setup mode 209 is turned off, the digital image 208 is passed to an algorithm 211 that calculates shifts in the fringe pattern 206. This output is split 212 to a real time output display 213 and is also written to a temporary file 214. At any time the user can save the data 215, which then writes the data to a permanent file 216.

BSI can detect changes in refractive index in real time. Therefore, it is a useful tool for measuring binding assays in real time. Also, BSI can be used to compare two samples for differences in refractive index, thereby indicating differences between the contents of the two samples.

Interferometric detection is amenable to high throughput assay methods, as the molecules, particles or cells do not require labeling with other reagents, such as fluorescent tags, thus requiring less processing of individual samples. The presence of the mass of the immobilized target or a signal due to a binding pair in solution, in embodiments where no binding moiety is immobilized, is detected directly as a function of interferometric signal and is robust under laser interrogation. The resulting signal is not susceptible to the photobleaching and loss of precision under long or repeated laser exposure of fluorescently labeled targets. Interferometric detection is a sensitive method of detection. Femtomolar levels of numbers of molecules can be detected and low picomolar (10-12) concentrations of target molecules can be detected.

An analyte in a sample can be detected in a sample in a number of ways. First, the interference patterns of a sample and a matched control can be compared. For example, a control sample should contain the same reagents and be contained in a container of the same dimensions as the test sample, but exclude the analyte. In this case, an important element that contributes to differences in the interference patterns will be differences in interaction between the analyte and the reagents in the two samples. For example, in a binding assay, differences between the concentration of an analyte between the two samples will be result in differences in amount of binding with a binding reagent, which, in turn, will result in differences in the interference pattern produced.

However, control and test samples may not be evenly matched. For example, a control plasma sample and a test plasma sample may have differences in various molecules that will result in differences in refractive index even if the concentrations of the analytes are the same. If analyte concentration differences contribute most to differences in refractive index, then this need not be an issue. However, these differences can be addressed in various ways. For example, a kit can provide reagents to construct a standard curve. Measuring results on the test sample against the standard curve provides an indication of the quantity of the analyte in the sample. Comparison of two samples, one with the reagents and one without, provides a measure of what contribution the presence of analytes makes to changes in refractive index. A test sample can be divided between two containers, one with reagents and one without, for this purpose. Moreover, for heterogeneous assays which employ sample vessels for which capture molecules have been selectively deposited in given probe regions, sample and experimental measurements can be conveniently performed within a single tube. In this approach, a sample of interest is selectively captured using capture molecules prudently localized within the probed region of the sample beam, while the reference beam interrogates a different region of the same vessel, which is devoid of extracted analyte. In this approach sample and reference measurements are performed on the sample matrix solution, variations in biological matrix, such as serological composition, ionic strength, and other bulk propertied can be compensated enhancing the signal to background.

The system can be used to determine the on- and off-kinetics of binding with a flowing system. In the flowing system, one molecule can be attached to the surface with chemistry. A running buffer is then flowed over the activated surface. Once the signal is stable, a second molecule that binds to the first is flown thought the system in increasing concentrations. When the sample interacts with the surface, there is an increase in signal until equilibrium is reached. When the running buffer is flowed back through, the bound molecules disassociate and the signal decreases and then equilibrates on the running buffer. For the reaction of the two molecules, an increase in signal is observed and then equilibrates. For this part of the curve, a ‘one phase exponential association’ equation is used [Y=Ymax*(1−exp(−K*X))] where K is the K observed. For the dissociation of the two molecules, a decrease in signal is observed until an equilibrium is reached. For this part of the curve, a ‘one phase exponential decay’ equation is used [Y=Span*exp(−K*X)+Plateau], where the K is the K off. The K on value is calculated by subtracting the K off from the K observed then dividing the value by the concentration of the binding ligand {Kon=(Kobs−Koff)/[ligand]}. The KD value is collected by dividing the K off by the K on [KD=Koff/Kon]. These equations assume one to one binding and that the concentration of one of the molecules is unchanged during the reaction. This is accomplished by the use of the flow as there is a constant amount of the same concentration being introduced into the channel.

D. Instrument with Continuous Injection

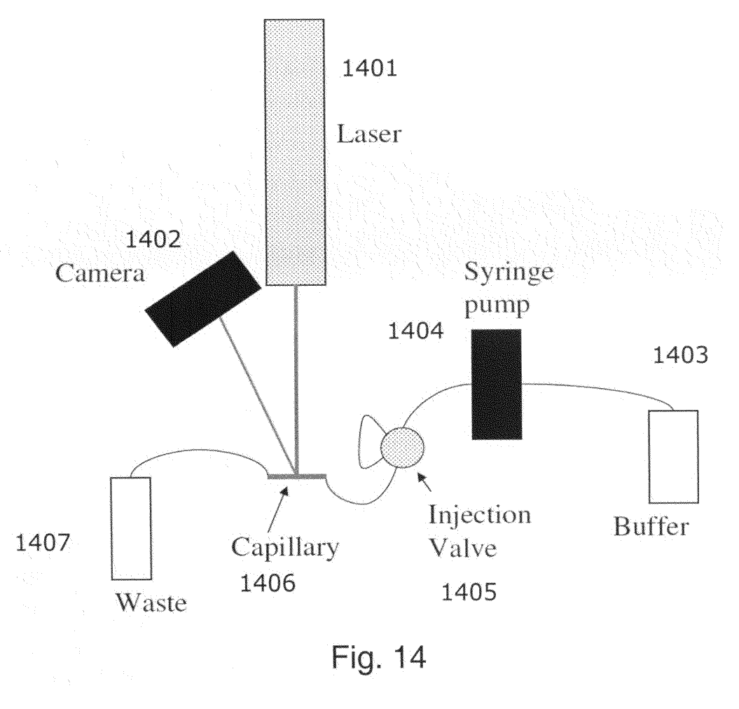

One version of the instrument allows for sample analysis in flowing streams. (See FIG. 14.) The basics of the instrumentation are the same; a coherent light source 1401 is directed onto a fluidic channel 1406, which produces a fringe pattern that is captured by a camera 1402.

A syringe pump (Cavro) 1404 is utilized with an injection valve to create a flowing system. The syringe pump pulls in a volume of liquid from a container 1403 which is then dispensed at desired flow rates. These rates can range from 10 microliters per minute to 0.5 microliters per minute, e.g., approximately 2.5 μL/min. The fluid passes through an injection loop and then the detection zone of the instrument. This provides a continuous flow of running buffer in the system. The injection loop can have a volume of 20 μL that can be changed based on the size and length of tubing used. The injection valve 1405 allows the injection of different samples without disrupting the flow of the system, as when in the load position the valve circumvents the loop allowing the running buffer to continuously flow. A sample is injected using a 250 μl analytical glass syringe into the loop. When the valve is switched to the inject position, the running buffer flows through the loop, pushing the injected sample into the detection zone. Thus the flow is never interrupted, aside from during the pump refill cycle.

The injected samples are pushed into the BSI instrument, which has a holder, which equilibrates the temperature of the fluid to a set point (typically 25° C.) by wrapping the capillary around a metal bobbin that is temperature controlled. The fluid is then pushed into the detection zone.

The detection zone is a small piece of capillary that the laser strikes. The small section of the capillary allows for surface chemistry to be performed on a large section and then cut into smaller sections for a heterogeneous experiment. After the fluid is analyzed, a waste tube is used to direct the sample into a waste container 1407.

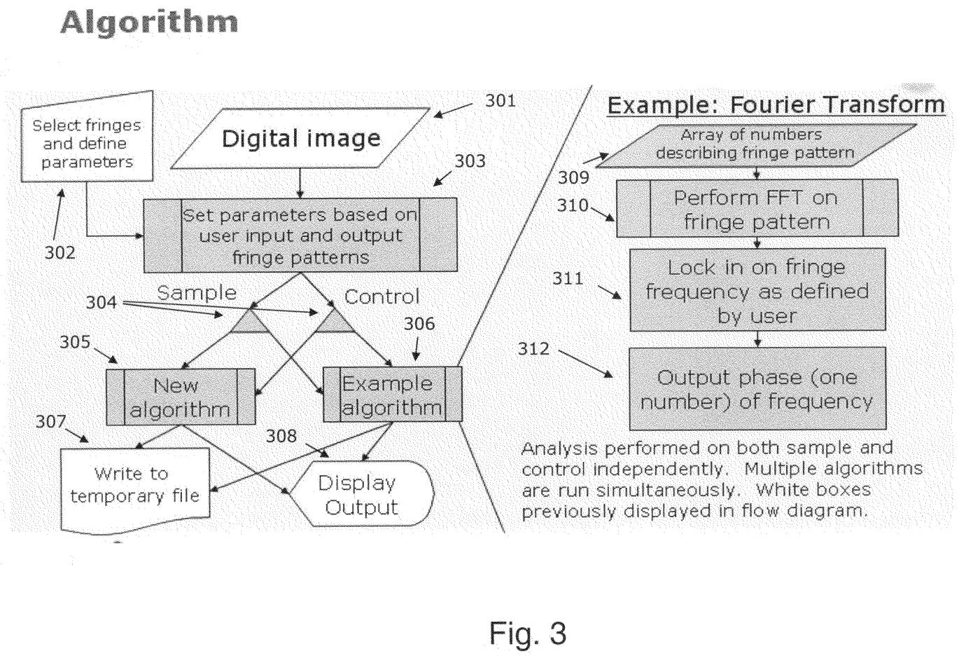

FIG. 3 shows a Fourier transform algorithm that transforms a digital image into a function that describes the image. Phase changes for the predominant spatial frequency in the Fourier transform over time can indicate shifts in the fringe pattern.

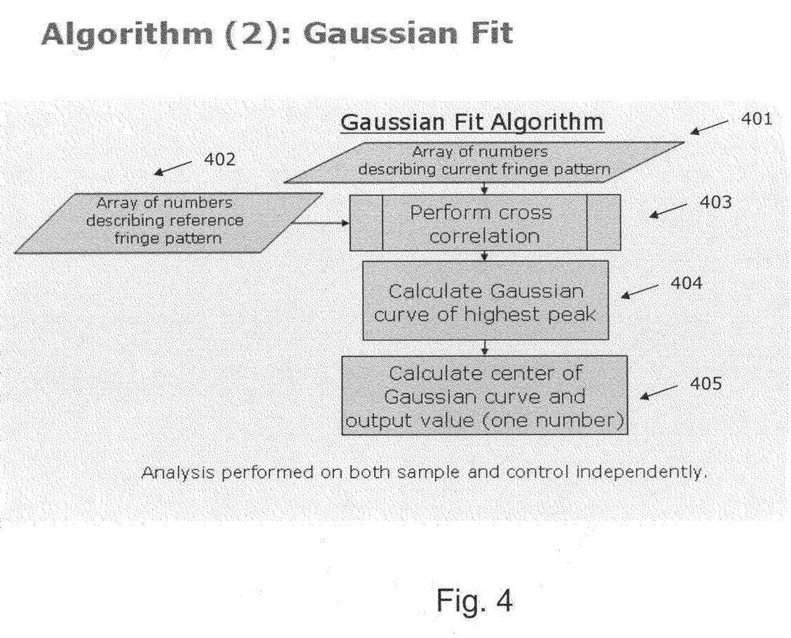

FIG. 4 shows the Gaussian fit analysis. A cross correlation is performed on a reference fringe pattern and a new pattern. A Gaussian fit is calculated from the highest peak of the cross correlation. The calculated center of the Gaussian fit is used to measure the pixel shift, which allows for sub-pixel shift detection.

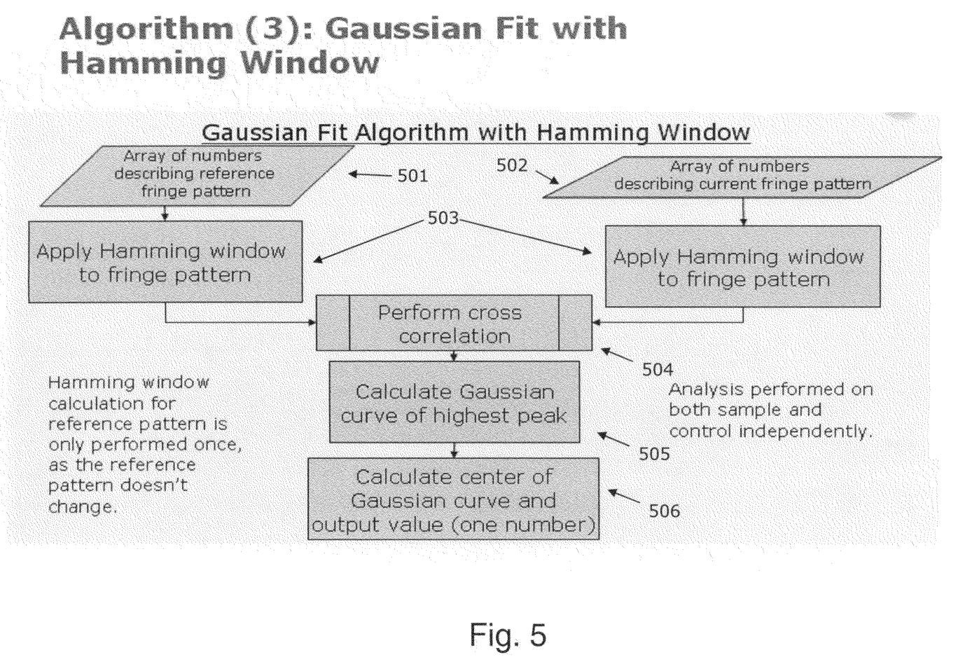

FIG. 5 shows the use of a hamming window, which is applied to the fringe pattern before the cross correlation is performed. Then a Gaussian fit of the cross correlation is used to determine the shift in the fringe pattern. The hamming window helps to minimize the noise.

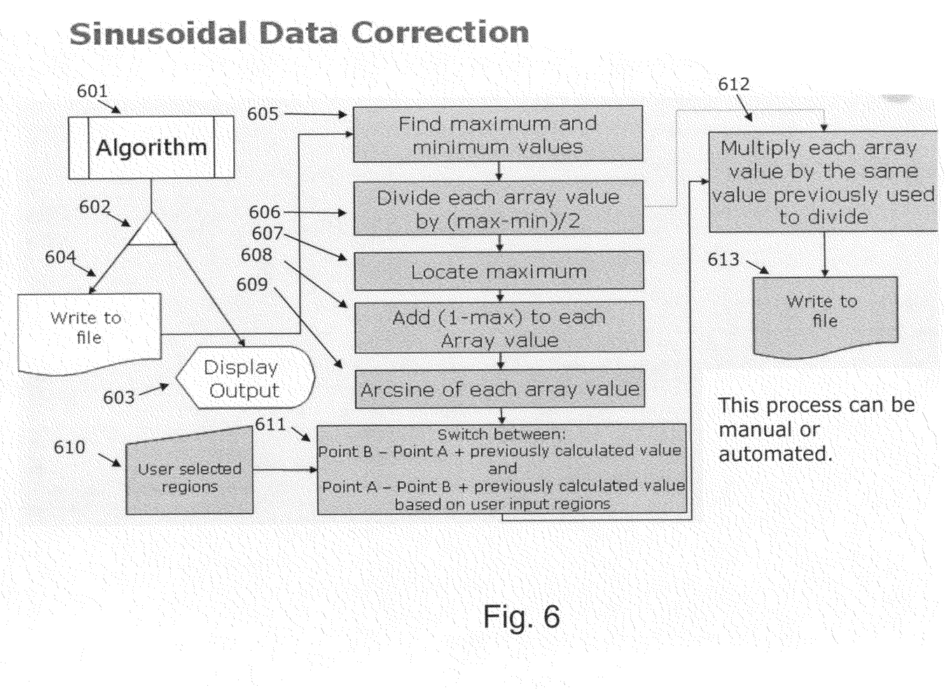

FIG. 6 shows a sinusoidal correction for data, which takes a set of data that has a sinusoidal output and unwraps the sine wave. Initially, it is required to divide the data so that it falls between −1 and 1. Next, an arcsine function is used. The sections of the data are then divided by the sharp peaks, and by using the equations, “Point B−Point A+previously calculated point” and “Point A−Point B+previously calculated point” on the different regions, it is possible to have the signal proceed in the same direction (unwrapping the sine wave into a line).

BSI can detect changes in refractive index in real time. Therefore, it is a useful tool for measuring biding assays in real time. Also, BSI can be used to compare two samples for differences in refractive index, thereby indicating differences between the contents of the two samples.

First, the interference patterns of a sample and a matched control can be compared. For example, a control sample should contain the same reagents and be contained in a container of the same dimensions as the test sample. For example, a control plasma sample and a test plasma sample may have differences in various molecules that will result in differences in refractive index even if the concentration of the analytes are the same. In an aspect, a system is provided for determining a characteristic property of a liquid that comprises: a device configured to detect a fringe pattern generated from a liquid; and a processor configured to receive information from the device, wherein the processor is configured to execute a set of instructions for processing the fringe pattern at more than one time by fitting the fringe pattern to a Gaussian distribution.

The processor can be a component of a computer system and the computer system can be configured to control the operation of the device. A signal analyzer comprising the processor, such as a computer or an electrical circuit, can be employed for analyzing the photodetector signals, and determine the characteristic property of the sample.

The signal analyzer can be a computer which, optionally, controls other aspects of the system. The computer functions to perform the calculations necessary to detect the fringe movement and output the data on the user interface. Moreover, the computer can function to store and retrieve method files which automate the performance of an assay or analysis, provides data analysis tools to determine binding profiles, qualitative measurements, and quantitative measurements, as well as providing a means to calibrate the system for total gain and output based upon a reference sample.

In an embodiment, the set of instructions when executed subject the fringe pattern to a Hamming window analysis prior to fitting the fringe pattern to a Gaussian distribution. The set of instructions can be a program code that when executed analyzes a series of fringe patterns.

In another embodiment, the processor is configured to execute a set of instructions that when executed compare fringe patterns at a first time to fringe patterns at a second time.

In some instances, the device has a pixel resolution and the comparison of fringe patterns at the first and second times has a sub-pixel resolution.

E. Container Mounting/Temperature Regulation

The device of this invention typically comprises a mounting adapted to receive the container and position it for interrogation by the coherent light source. The mounting can be removable from the frame of the device. The mounting can be attached to an optical bench that comprises other components of the optical system. The mounting can comprise a fastener to fasten the container to the mounting. If the container is a tube, the mounting can comprise, for example, a clip or set of clips, a surface with an indentation adapted to receive the tube, in which it can rest, an adhesive material, or a holder in which the container is inserted and held, e.g., a cylinder in which a tube is slid within and retained, a flat mounting stage on which a chip is locked into position. In certain embodiments the mount is in thermal contact with a temperature control assembly such as a Peltier device to insure homogeneous control of temperature as required to perform high sensitivity BSI measurements (+/−1-5 millidegree C.). See, for example, U.S. patent publication 2009-0185190.

A container of the invention can be adapted and configured to fit snugly within a holder. The container can be held in place by a positioner, such as a metal plate with tightening screws. The container can be manually inserted into the holder or cartridge. The container can be oriented so that gravitation force maintains the separation of the solid and liquid material, e.g., the liquid is oriented on top of the separated solid material. In an embodiment, the container is disposable while the holder can be used for numerous different chips with a device of the invention. A holder retention mechanism can be used to firmly hold the chip in the holder along the axis of the mechanism. The container and/or the thermal subsystem can be affixed to a translation stage that allows adjustment of the chip relative to the laser beam. For example, the container can be tilted slightly (for example, approximately)₇° so that the backscattered light from the sensing area of the container can be directed onto the photodetector.

In experiments that involve comparing the interference pattern between two samples (e.g., a test and control sample), the samples can be measured simultaneously or in sequence. In simultaneous measurements the two samples can be loaded onto the interferometer and a beam splitter can split the laser beam and direct it to each of the two samples. Alternatively, the beam can be made wide enough so that a single beam covers both fluid compartments. In one embodiment, the first and second samples are comprised in different containers, e.g., tubes, and one tube is tilted or rotated, e.g., 3° to 7° with respect to the other tube. This results in the interference signal from each container being directed to different parts of the detector so that they are distinguishable.

In another embodiment, the first and second samples are located within a single tube, where the first sample represents a region of the sample container that contains a selectively deposited binding molecule for extraction and subsequent analysis of a target of interest, and where the second or reference sample represents a region of the sample container that is free of binding molecule, or moreover is coated with a specific passivating agent to minimizing unwanted non-specific binding of the target of interest.

Sample can be introduced into the container by any method known. For example, the sample can be introduced manually using a syringe, e.g., manual pipetter. Also, sample can be introduced into the container using a fluidics robot, such as any commercially available robot, e.g., from Beckman or Tecan.

### V. Methods of Detecting Analytes in a Sample

The ability to collect a biological sample directly from a subject and analyze it by back scattering interferometry in the same container allows a variety of clinical assays to be carried out easily. In certain embodiments, the assays are carried out in near patient (e.g., at the patient's location such as in a hospital room or doctor's office) or point-of-care conditions. In other cases, the assays are carried out in a central location with other samples, e.g., a laboratory. The invention provides an ability to incubate a sample, to centrifuge a sample to separate insoluble material, and to analyze the fluid, without having to change sample containers for the purposes of the manipulation. One aspect of the invention that makes this possible is adaptations in the interferometer to mount the collection container, itself.

Several kinds of assays to detect analytes are contemplated by this invention. They include, without limitation, (1) homogenous or heterogeneous binding assays to detect and/or quantify an analyte and (2) enzymatic assays to detect and/or quantify an analyte.

A variety of assays are contemplated by this invention. These include, for example, reactive titers, infectious diseases, drugs of abuse, sepsis, oxygen monitoring, detection of biomarkers of disease (e.g., proteins) molecular biological assays such as SNP analysis, STTR analysis, hybridization analysis for genotyping or gene expression, per analysis, allelotyping, haplotyping, as well as monitoring of enzymatic reactions.

Alternatively, a difference in titer of certain analytes compared with a control also can be detected by BSI.

A. Binding Assays

An analyte can be detected in a sample through a binding assay with a binding reagent. A binding reagent can specifically bind to the target analyte.

Any analyte that has a binding partner can be detected by including the binding partner in container. Binding between the binding partner and the analyte will result in a change in refractive index that can be detected by BSI. For example, the analyte could be a component of an infectious agent. Alternatively, it could be a biomarker for a disease, such as cancer. Any molecule that can be captured can be detected by BSI.

In a homogenous assay, the binding partner is free in the compartment and is taken into solution upon contact with the sample. In a heterogeneous assay, the binding reagent is immobilized to the wall of the compartment. In a specialized form of heterogeneous assay, the binding agent is selectively deposited upon a distinct region of the compartment wall, while all other areas are devoid of the binding agent, allowing for sample and reference measurements to be simultaneously performed in a single sample compartment. Methods for immobilizing a binding reagent to a wall of a compartment are well known in the art. For example, for any surface with available reactive groups, such as glass, the reactive groups can be coupled to a silane containing moiety by using a reactive compound such as amino-propryl-triethoxy silane or mercapto-amino-propyl-triexthoxy silane. A bifunctional coupling agent can then be employed to covalently attach to the silane layer and subsequently couple its other end to a target biomolecule, tethering that biomolecule to the surface. Exemplary bifunctionial linkers include but are not limited to, succinimidoalkylbenzaldehydes, dimethyl dithiobispropionimidate, N-[gamma-maleimidobutyryloxy]succimide ester, and N-[gamma-malaeimidobutyryloxy]sulfosuccinimide ester. Coupling to the desired target biomolecule is achieved via reaction between the terminal group of the bifunctionial linker and a companion reactive group of the biomolecule such as an amine, a hydroxide, a sulfhydryl, a carboxyl, and so on.

Analytes in the blood that can be detected in binding assays include, for example, pathogneumonic antibodies indicative of infections disease, autoimmune disease, or cancer; surface antigens or liberated proteins from infectious elements such as parasites, bacteria, viruses, and molds; surface antigens or liberated proteins from host neoplasms; specific host response proteins to tissue damage, necrosis, apoptosis; specific host proteins spawned as the result of general inflammatory response damage as associated with autoimmune disease, rheumatoid arthritis, osteoarthritis, cancer, ethanol toxicity, therapeutic agent toxicity, drug abuse, and/or infectious disease; liberated proteins associated with ischemia and tissue damage as in cardiomyopathies, drugs of abuse and their metabolites, therapeutics and their metabolites; and so on.

Binding agents include, for example, aptamers, thioaptamers, double-stranded DNA sequence, peptides and polypeptides, ligands and fragments of ligands, receptors and fragments of receptors, antibodies, fragments of antibodies (e.g., a single chain antibody, an Fab, Fab′ F(ab′) 2 fragment) or hybrid antibodies and polynucleotides. The binding reagent can also be a member of other types of binding pairs such as biotin-avidin; apo-protein:cofactor; lectin-saccharide (or polysaccharide); lectin-cell; IgG antibody Fc portion with protein A or protein G; enzyme-enzyme substrate; sense-antisense nucleic acid sequences such as DNA:DNA, RNA:RNA; DNA:RNA, DNA fragments or other nucleic acid sequences; enzyme-enzyme inhibitor; receptor-ligand; protein-protein receptor; protein subunit-protein subunit; or lipid-lipid.

B. Enzymatic Assays

Enzymatic assays typically are time course assays. In such assays, one measures differences in refractive index in a sample over time. Differences indicate the action of the enzyme on the analyte. One example of an enzymatic assay is enzymolysis.

In one assay, the tube is provided with substrates for enzymes in the sample. For example, typical enzymes detected in the blood of clinical interest include alkaline phosphatase, amino transferases (e.g., aspartate transaminase, alanine transaminase, gamma glutamyl transferase), lactate dehydrogenase, and creatinine kinase.

In this type of assay, the container is provided with a substrate that is cleaved by a serum protease, such as alkaline phosphatase activity upon a phosphopeptide, phosphoprotein, phosphorylated nucleic acid or phosphorylated polynucleic acid. In this type of assay a general assessment of serological enzymatic activity against a number of serum proteases could be assessed as part of a diagnostic regimen.

Other enzymatic assays are used to detect the presence of a nucleotide sequence in DNA. For example, in PCR, primers, nucleotides and a polymerase are used to amplify a sequence within a DNA sample. This typically involves thermal cycling, in which each cycle amplifies the target sequence. Measurements can be taken after each cycle. Again, changes in refractive index result from polymerization reactions which, in turn indicate the presence of the target sequence. Other methods of DNA sequence detection are known in the art. One of these is detection by ligation, in which probes that hybridize to adjacent sequences are provided with a ligase. If the target sequence is present, the probes will hybridize adjacent to one another and the ligase will ligate the two probes. This change can then be detected.

C. Sample Assays

The sample collection process for a serological or hematological test can proceed as follows:
1. A finger stick is performed using a typical disposable lancet.
2. The capillary tube is employed to receive a given volume of whole blood from the finger stick wound. The blood enters the tube via capillary action.
3. The top portion of the tube is plugged by inserting it into a wax plane or plane of other compliant sealant. Alternatively the tube is fashioned with an adapter that allows for closing one side of the tube with a cap and the other side of a tube with a plug.
4. The tube is placed in a holder to allow it to incubate at a predetermined temperature over a predetermined period of time.
5. Optionally, the tube is placed into a modified laboratory mixer that allows for agitation of the solution during the incubation period.
6. The tube is placed into a standard hematocrit centrifuge and spun for the requisite period of time to divide the volume into roughly three regions: RBC, buffy coat, and plasma.
7. The tube is placed into a small table top or portable BSI device, and the assay is read by probing the relatively transparent plasma region of the tube.

A typical assay can be performed as follows:
1. A negative control tube is inserted into the BSI device. A background measurement is performed.
2. A clinical specimen tube is inserted into the BSI device. The measured signal is compared to that obtained in the control to allow for the relative quantitative measurement of the target analyte.
3. Alternatively a two beam BSI instrument which allows the simultaneous illumination of both the sample and reference capillary can be employed. In this case the clinical specimen and the control would be evaluated at the same time.
   a. A single beam from a coherent source, preferably a diode laser, illuminates both capillaries, touching each other at the point where the beam impinges on them, producing two fringe patterns. Each capillary would be tilted 3°-7° degrees off of normal so that the backscattered fringe patterns are spatially separated.
   b. The two capillary configuration output is a sample vs. reference measurement and provides enhanced S/N.
   c. This configuration also greatly reduces external temperature control requirements as both capillaries in the same environment, allowing less power consumption, etc.
   d. This configuration also allows for automatic laser intensity instability compensation, particularly important when employing a photon flux-based sensing approach.
4. Quantification of response or analyte concentration come from interrogation of the fringe pattern, which can be done by several methods, but most commonly, involves measuring the shift in position. Then the output can take the form of Sample−Reference, S−R/S+R or other signal extraction algorithms.

This invention contemplates both homogeneous and heterogeneous assays. In a homogeneous assay, the binding agent, e.g., a binding partner, is in free solution to interact with the analyte. In a heterogeneous assay, the binding agent is tethered or otherwise attached to the inside surface of the compartment to interact with the analyte. In certain embodiments of a heterogeneous assay, the surface of the compartment is patterned such that a binding agent is bound at a specific or predetermined location on the compartment surface, where interaction with analyte can be interrogated. For example:
1. The sample is introduced into a tube that contains patterned deposits of binding partners (e.g., antibodies, one for each analyte of interest) and specified regions devoid of these agents which act as controls.
2. The sample is allowed to incubate.
3. The sample is then processed by spinning to make the patterned region devoid of insoluble particulates or the sample can be then pushed out of the tube using an appropriate buffer wash solution.
4. After processing, a single wide beam, or multiple beams, are used to illuminate each specified region. Detected fringe pattern differences are used to compare assay and control signals.

For each of the above, it is possible to perform analysis with simultaneous controls or compare results against controls performed as a separate experiment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

U.S. Pat. No. 5,325,170
Bornhop D J: U.S. Pat. No. 6,381,025: Inteferometric Detection System and Method. In USPTO (ed): USPTO. USA, Texas Tech University, 2002:1-7.
U.S. Pat. No. 6,559,947
Bornhop D J: U.S. Pat. No. 6,809,828: Universal Detector for Biological and Chemical Separation or Assays Using Plastic Microfluidic Devices. In USPTO (ed): USPTO. USA, Texas Tech University, 2004.
Bornhop D J: U.S. Pat. No. 7,130,060: Refractive Index Determination by Micro Interferometric Reflection Detection. In USPTO (ed): USPTO. USA, Texas Tech University, University of Denmark, Riso National Laboratory, 2006.
U.S. application Ser. No. 12/587,112 (filed Sep. 30, 2009, Chang)
U.S. patent publication 2006-0012800
U.S. patent publication 2009-0185190
International application WO 2004/023115
International application WO 2006/047408
International application WO 2009/039466
Markov D A, Swinney K, Bornhop D J: Label-free molecular interaction determinations with nanoscale interferometry. J Am Chem Soc 2004; 126:16659-16664.
Swinney K, Markov D, Bornhop DJ: Chip-scale universal detection based on backscatter interferometry. Anal Chem 2000; 72:2690-2695.
Sorensen, H. S.; Larsen, N. B.; Latham, J. C.; Bornhop, D. J.; Andersen, P. E., “Highly sensitive biosensing based on interference from light scattering in capillary tubes”, Applied Physics Letters 89(15) 2006.
Bornhop, Darryl J.; J. C. Latham, Amanda Kussrow, D. A. Markov, Richard D. Jones and Henrik S. Sørensen, “Free-Solution, Label-Free Molecular Interactions Studied by Back-scattering Interferometry”, Science 21 Sep. 2007: Vol. 317. no. 5845, pp. 1732-1736
Dendane et al., Lab Chip 2008, 8:2161

What is claimed is:

1. A method comprising:
a) introducing a sample into a container, wherein:
i) the sample comprises a liquid and insoluble material; and
ii) the container is adapted for analysis of the liquid therein by back-scattering interferometry;
b) separating the liquid from the insoluble material within the container; and
c) analyzing the liquid while in the container by back-scattering interferometry.

2. The method of claim 1 wherein the sample is blood.

3. The method of claim 2 wherein the liquid is serum or plasma.

4. The method of claim 1 wherein the container comprises a solid substrate comprising a channel into which the sample is introduced.

5. The method of claim 1 wherein the container contains an anti-coagulant.

6. The method of claim 1 wherein the container contains a reagent for an assay for an analyte in the sample.

7. The method of claim 6 wherein the reagent is a binding partner for the analyte.

8. The method of claim 6 wherein the reagent is immobilized to an internal surface of a compartment of the container.

9. The method of claim 8 wherein the internal surface is patterned so that the reagent is attached only to a portion of the surface and that portion is positioned in a sensing area that is interrogated by a laser during back scattering interferometry.

10. The method of claim 9 wherein the method comprises detecting position of a fringe pattern generated at a location where the reagent is immobilized and at a location is where the reagent is not immobilized and comparing the positions.

11. The method of claim 6 wherein the reagent is an enzyme.

12. The method of claim 6 wherein the assay is PCR and the container contains amplification primers, polymerase and nucleotides.

13. The method of claim 6 wherein the assay is a ligation assay and the container contains a ligase.

14. The method of claim 6 wherein a plurality of reagents are immobilized at different specific locations on an internal surface of the compartment.

15. The method of claim 6 wherein the reagent is free for entering solution upon introduction of a liquid sample into the compartment.

16. The method of claim 1 wherein the container is a chip.

17. The method of claim 1 wherein the container is a tube.

18. The method of claim 17 wherein the tube comprises borosilicate, fused silica or plastic.

19. The method of claim 17 wherein the tube has an internal diameter of 0.05 mm to 2.0 mm.

20. The method of claim 17 wherein the tube has a bore with at least one flat face.

21. The method of claim 1 wherein introducing comprises collecting blood from a subject into a capillary tube and sealing at least one end of the capillary tube.

22. The method of claim 21 wherein separating comprises centrifuging the capillary tube.

23. The method of claim 1 wherein introducing comprises providing a capillary tube fitted on one end with a holder comprising a hole that securely fastens to the end of the capillary tube and a well for accepting liquid and passing it into the capillary tube.

24. The method of claim 1 further comprising incubating the sample or agitating the sample after introduction.

25. The method of claim 1 wherein separating comprises centrifugation or sedimentation.

26. The method of claim 1 wherein analyzing comprises detecting the presence of an analyte in the liquid.

27. The method of claim 1 wherein analyzing comprises engaging the container with a holder of a back-scatter interferometry device in a position so that the container can be interrogated by a laser of the device.

28. The method of claim 1 wherein analyzing comprises comparing a signal received from the liquid with a signal received from a control liquid.

29. The method of claim 1 wherein analyzing comprises simultaneously illuminating the liquid and a control liquid with a single beam from the coherent light source and comparing the signals.

30. The method of claim 1 wherein back-scattering interferometry comprises directed a coherent light beam at the fluid in the container and back scattered light.

31. The method of claim 30 wherein the coherent light is laser light.

32. A method comprising performing back-scattering interferometry analysis on a sample wherein the sample is stationary at the time of analysis.

33. A method comprising:
a) collecting a liquid sample from a subject into a sample collection container containing a compartment adapted for analysis by back-scattering interferometry; and
b) analyzing the liquid while in the sample collection container by back-scattering interferometry.

34. A device comprising:
a) a coherent light source;
b) sample container for receiving a sample, wherein the container is configured for analysis of a sample therein by back-scatter interferometry when interrogated by coherent light from the coherent light source, and wherein the container is further configured to prevent flow of a sample in the container during analysis; and
c) a detector to detect back-scattered light.

35. The device of claim 34 wherein the container is configured to centrifuge a sample contained in the container when the container is not engaged with the device.

36. The device of claim 34 wherein the container comprises at least one stop or valve that prevents fluid flow.

37. The device of claim 34 wherein the container comprises a reagent for performing an assay on an analyte.

38. The device of claim 37 wherein the reagent is immobilized on an internal surface of the container.

39. The device of claim 37 wherein a plurality of different reagents are immobilized at different specified locations on an internal surface of the compartment.

40. The device of claim 39 wherein the container is patterned with areas to which the reagents are immobilized and at least one area to which no reagent is immobilized.

41. The device of claim 40 wherein the reagent is immobilized by photolytically cleaving a protecting group attached to the surface to expose a reactive group and coupling the reagent to the reactive group.

42. The device of claim 40 wherein the reactive group is an isocyanate.

43. The device of claim 37 wherein the reagent is free for entering solution upon introduction of a liquid sample into the compartment.

44. The device of claim 34 wherein the coherent light source comprises a laser.

45. The device of claim 34 further comprising a centrifuge adapted to engage the container, to centrifuge the container while engaged, and to position the container for analysis.

46. The device of claim 45 wherein the container contains a channel and is adapted to receive a sample into the channel through capillary action, wherein at least one end of the channel is closed when a sample is collected to allow centrifugation.

47. The device of claim 46 wherein the container comprises a hematocrit tube.

48. The device of claim 46 wherein the container comprises a chip.

49. The device of claim 46 further comprising a second holder adapted to engage a second container in a position to be interrogated by an undivided beam of coherent light from the light source.

50. The device of claim 49 wherein the containers are positioned with respect to one another so that the detector detects the fringe pattern from each container distinctly.

51. A kit comprising:
a) a device comprising:
i) a coherent light source;
ii) a holder adapted to engage a container in position so that a liquid in the container can be interrogated by coherent light from the coherent light source to produce a back scattered light; and
iii) a detector configured to detect the back scattered light; and
b) a container comprising a reagent for assaying for an analyte in the container, wherein the container is adapted to engage the holder.

52. The kit of claim 51 wherein the container is a hematocrit tube.

53. The kit of claim 51 wherein the container is a capillary tube of fixed length.

54. The kit of claim 51 wherein the reagent comprises a binding partner for the analyte.

55. The kit of claim 54 wherein the binding partner comprises a polynucleotide, a polypeptide or a small organic molecule.

56. The kit of claim 51 wherein the reagent is immobilized on an internal surface of the compartment.

57. The kit of claim 56 wherein the reagent is locally immobilized on the internal surface of the photonicly probed region of the compartment.

58. The kit of claim 51 wherein the reagent is free for entering solution upon introduction of a liquid sample into the compartment.

59. The kit of claim 51 further comprising:
c) a sealant for sealing or a cap for closing an opening in the container.
